(19) United States
(12) Patent Application Publication
(10) Pub. No.: US 2020/0337639 A1
(43) Pub. Date: Oct. 29, 2020

(54) Wearable Exercise Assessment System

(71) Applicant: Incus Performance Limited, Leicestershire (GB)

(72) Inventors: Christopher James Ruddock, Leicestershire (GB); Heinz Lugo, Leicestershire (GB); Dimitrios Pantazis, Leicestershire (GB)

(21) Appl. No.: 16/761,143

(22) PCT Filed: Nov. 5, 2018
(86) PCT No.: PCT/GB2018/053209

§ 371 Date: May 1, 2020

(30) Foreign Application Priority Data
Nov. 3, 2017 (GB) 1718273.4

(51) Int. Cl. A61B 5/00; A61B 5/11; A63B 24/00; G16H 20/30; G16H 40/63; G09B 19/00

(52) U.S. Cl. CPC A61B 5/6831; A61B 5/486; A61B 5/6822; A61B 5/6823; A61B 5/1121; A61B 5/0205; G16H 20/30; G16H 40/63; G09B 19/0038; A61B 2562/0219; A61B 5/1112; A63B 24/0062

(57) ABSTRACT

A wearable exercise and performance assessment system has a harness adapted to be attached to the torso of a wearer. The harness comprises a mount for releasably mounting an electronics module housing at a predetermined location on the torso. The electronics module housing comprises motion sensors. A processor processes received sensor data signals and outputs a resulting feedback signal to a performance feedback device. The processor comprises a sensor signal receiver arranged to receive motion sensor data signals over time from the motion sensors and to log the received signals with a synchronized time signal. One or more of the received sensor data signals is processed to identify data features therein indicative of repeated movements by the wearer and to delineate each repetition thereof. The processor applies the repetition delineation to one or more further received motion sensor data signal to determine a performance parameter for each individual repetition thereof.

### CROSS-REFERENCE TO RELATED APPLICATION(S)

This application is a national stage application of International Application Patent Serial No. PCT/GB2018/053209, filed Nov. 5, 2018, which claims priority to United Kingdom Application Serial No. GB 1718273.4, filed Nov. 3, 2017, the entire disclosures of which are hereby incorporated by reference.

### TECHNICAL FIELD

The present disclosure concerns wearable sensor systems and associated processing equipment for assessing the effectiveness of exercise, including—but not limited to—competitive sport.

### BACKGROUND

Over recent years there has been a proliferation of fitness trackers in the form of wrist watches and the like arranged to monitor the level of general activity of a wearer. Such devices have a motion sensor and a processor to log rudimentary parameters attributed to exercise in general, or motion attributed to specific activities, such as walking, running, cycling or the like. It is known that such devices may have additional sensors for sensing heart rate and/or a GPS signal receiver to provide further data inputs that can be used to either improve the accuracy of motion/activity monitoring or else allow a wider range of parameters to be reported to the wearer.

However such devices are for general purpose use and do not provide outputs that help the wearer or a coach assess and improve technique. To this end, there have been proposed in the art a number of dedicated devices focussing on specific competitive sports to monitor a set of user movements.

EP2128724 discloses a watch device for use when swimming, which comprises a satellite signal receiver and a processor, and is able to better synchronise location based on the wearer's arm movement in conjunction with satellite location data.

A number of further patent publications disclose techniques for counting laps and/or strokes based on monitoring wrist/arm motion whilst swimming.

WO 2010113135 discloses a device worn by a swimmer using a chest strap. A three-axis accelerometer is arranged such that the axes are aligned with forward, lateral and vertical orientations for the wearer's body when swimming. This alignment is used to identify different swimming strokes as well as counting strokes and identifying turns made by the swimmer. However the device is not able to provide deeper insight into technique or the individual parameters that contribute to improved performance, nor provide physical feedback to the athlete in any particular time frame.

Whilst software applications have been developed to provide additional feedback to users to help improve technique, the ultimate usefulness of the feedback is strongly related to the quality of the data captured by the sensors. For example, it is known in swimming applications to use the so-called ‘SWOLF’ metric, which takes into account both stroke count and time/speed, as a measure of stroke efficiency. However the use of simplistic stroke count, distance and time parameters means that the user has no guidance as to how to improve stroke efficiency.

When training as a team, different athletes with different techniques may achieve similar results and so simple metrics of the type described above will offer little insight to a coach in understanding what aspects of technique to work on for each individual.

US2003/0138763 discloses a system for detecting, tracking, displaying and identifying repetitive movement during swimming. That document discloses a method of processing the signals of a two-dimensional movement sensor arrangement to determine indicators for stroke identification, stroke count, breathing pattern, turns and ‘stroke signature’. A received signal is correlated with a normal/calibrated signal for the user to identify events such as breathing or turns.

There is a limit to the complexity of information that an athlete can readily appreciate during training. However certain aspects of technique are best corrected in real time and so there is often a conflict between the need for deep and detailed analysis and the timeliness of the results of such analysis. Conventional high-speed, video-based measurement systems for technique analysis are costly to procure and operate, relatively slow to produce results and do not accommodate scaling of the technology for multiple individuals training in groups or teams.

It is an aim of the present disclosure to provide a wearable sensor system and associated data processing that can provide deeper analysis of technique for competitive sport. It may be considered an additional or alternative aim to provide a system that can accommodate the needs of individuals, groups and/or coaches.

### SUMMARY

According to the present disclosure, there is provided a wearable exercise assessment system comprising a harness adapted to be attached to the torso of a wearer and an electronics module comprising a housing having a plurality of motion sensors, a processor—optionally within the housing—for processing received sensor data and outputting a resulting feedback signal to a feedback device of the system, wherein the harness is arranged to hold the housing at a predetermined location on the torso of the wearer and comprises a mount for releasably mounting the module housing to the harness.

The harness may comprise an electrical connector for establishing an electrical connection with the electronics module when mounted thereon.

The harness may extend over a portion of the torso in use and may comprise the feedback device and/or a further sensor for communication with the electronics module when connected thereto.

A feedback device may be provided on, or connected to, the electronics module and/or harness. The feedback device may comprise any, any combination or all of: a haptic device; one or more light; and, one or more audio output circuit or speaker. A plurality of lights, e.g. light emitting diodes, may be provided. The individual lights and/or haptic motors may be individually and/or collectively controllable by the processor. The one or more light may be visible to an onlooker and/or a vision inspection system.

The processor may control operation of the feedback device according to one or more sensor data parameter or a parameter derived by the processor from the sensor data, e.g. in real-time. One or more parameter threshold or event (e.g. a max/min threshold, rate of change threshold or change of state event) may be used to trigger operation of the feedback device and/or change an output state of the feedback device.

The processor may be selectively configurable/programmable to operate the feedback device according to one or more parameter and/or one or more parameter threshold selected by a user. A user selection may be made using one or more further user device, e.g. a mobile communication device, such as a smartphone, tablet, or similar.

The movement sensors of the module may comprise one or more inertia sensor. The movement sensors may comprise a multi-axis movement sensor or a plurality of multi-axis movement sensors. The movement sensor may comprise a plurality of different sensor types. The movement sensors may comprise any, any combination or all of: one or more accelerometer; one or more gyroscope; and/or or more direction sensor, such as a compass/magnetometer.

One or more further sensor, such as an orientation sensor may be provided with/in the housing. A magnetic field sensor may be used.

The electronics module typically comprises a power source, such as a battery.

The electronics module may comprise a self-contained unit having a core set of sensors, i.e. such that the electronics module can operate autonomously and/or independently of the harness. However the coupling with a harness allows the electronics module to be used as part of a bespoke system that can be tailored to a specific sport or activity, e.g. by selection of a harness tailored to that activity. Thus a common electronics module could potentially be used with a variety of different harnesses as required. Additionally or alternatively, the electronics module on a harness may be replaced, e.g. hot swapped, with another harness as necessary.

The harness may comprise an adhesive, e.g. an adhesive region or layer. The adhesive may be provided on an outer layer/surface of the harness. The adhesive may be provided for attaching to a wearer's skin.

The harness may comprise a continuous sheet material. The harness may comprise a flexible, e.g. polymer, sheet. The harness may comprise a laminate structure.

The harness may comprise a plurality of layers. One or more layer may comprise an insulating layer, which may comprise an electrical insulation layer and/or thermal insulation layer.

The, or each, further sensor and/or feedback device of the harness may be spaced from the mount, e.g. by an elongate conductor. One or more electrical conductor may be embedded in a layer or between adjacent layers of the harness.

The harness may comprise a one or more haptic feedback device, e.g. a plurality of feedback devices at spaced locations on the harness. Additionally or alternatively, the harness may comprise one or more light, e.g. an array of individually controllable lights, such as LEDs.

The harness may be curved in form. The harness may be shaped so as to provide a collar arranged to extend at least part way around the wearer's neck, e.g. lower neck.

The harness may extend over each clavical/collar bone of a wearer. The harness may extend over the trapezius muscle region of a wearer, e.g. the superior or intermediate muscle region.

The harness may comprise one or more limb. The harness may comprise a limb or limb portion extending on opposing sides of the mount. The mount may be centrally positioned on the harness. The harness may be symmetrical about the mount and/or a central axis. The harness portion to one side of the mount and/or central axis may be substantially a mirror image of the other side.

The mount/module may be arranged to be located on the upper torso of a wearer, e.g. at the base of a wearer's neck. The mount/module may be mounted between a wearer's shoulder blades.

The mount/module may be centrally located on the wearer's body, e.g. on the sagittal plane. The mount/module may be aligned with the wearer's spine e.g. at the upper thoracic spine or lower cervical spine. The mount/module may be located in the region of the interface between the upper thoracic spine and lower cervical spine.

The module housing may depend from and/or extend beyond the mount/harness when mounted thereto, e.g. beyond a perimeter of the harness.

The harness may comprise a plurality of sensors, e.g. located on opposing sides of the mount. The harness may comprise three or four or more sensors.

The harness sensor(s) may comprise any, or any combination, of motion, environmental and/or physiological sensors. A physiological sensor may comprise a heart rate, blood oxygenation or body temperature sensor. An environmental sensor may comprise a barometric pressure sensors or temperature sensor, etc.

The harness may comprise a (micro)controller and/or a plurality of electronic control devices/circuits to manage the communication from or to any embedded sensors or feedback methods.

The housing may comprise body and tail regions. The housing may taper towards the tail region. The housing may be generally teardrop shaped in plan.

The housing may comprise an electrical and/or mechanical connector on an underside of the body region.

The housing may or may not be convex in form on its upper/outer surface. An underside of the housing may comprise a concave portion or form.

The upper/outer surface of the housing may comprise visual indicia, e.g. for visual identification by eye or by visual inspection apparatus. The visual indicia may comprise a contrasting colour, tone, lightness or brightness from a surrounding portion of the surface or the remainder of the surface.

The upper/outer surface of the housing may comprise one or more light. The one or more light may be controlled by the processor to provide varying colour and/or brightness and/or lighting pattern output.

The use of indicia on the outer surface of the housing allows it to be readily seen by a coach or visual inspection unit.

The processor may process the received sensor data in real time to determine one or more exercise parameter. The processor may or may not determine forward speed and/or velocity based on the motion sensor data, e.g. based on the motion sensor data alone. The processor may determine a relative change in forward speed and/or velocity.

The processor may determine rotation, e.g. of the module about a sagittal, fontal and/or vertical axis of a wearer.

The processor may determine relative sensor movement or acceleration between the movement sensor(s) of the module and the one or more further sensor of the harness.

The processor may comprise a plurality of processors, a first processor arranged to coordinate receipt of raw sensor signals from the motion sensors with a common timing signal. Said first processor may be provided in the module housing. Said first processor and the motion sensors may or may not be provided as a common chip. One or more further processor may further process the output of the first processor to generate the one or more resulting feedback signal. The further processor may be provided in the module housing or remotely, e.g. in communication with the module over a local area or wider area network connection.

The processor may comprise a data fusion module for combining incoming motion sensor signals, e.g. comprising any combination of acceleration, angular velocity and orientation/magnetic field signals. The data fusion module may output any or any combination of linear acceleration, gravity vector, Euler angles, yaw, pitch, roll and/or rotation matrix signals.

The motion sensor signals, e.g. the output of the data fusion module, may be processed to identify cyclic signals, e.g. according to identification of one or more repeating feature in the signals. Each of the signals may be partitioned into individual cycles based on an identified periodicity/frequency. Data features, such as max, min, zero-crossings of the motion signals, or gradients, thereof, may be determined.

The motion sensor signals, e.g. the output of the data fusion module, may be processed to identify segments of the motion signals corresponding to different aspects of the exercise performed by the user.

Statistical feature extraction may be performed on either or both of the individual cycles and segments identified by the processor, e.g. for the entire signal duration or a portion thereof.

A relative change in speed and/or acceleration, e.g. in one or more direction, may be determined for each cycle and/or segment.

Different categories of sensor/exercise parameters and/or data sets may be identified and/or managed by the processor. A first category may comprise data/signals to be processed in real time and/or used to provide output by the feedback device. A second category may comprise data/signals to be processed in near-real time, e.g. for analysis/communication during an instance of exercise. A third category may comprise data/signals to be processed and/or analysed with a time-delay, e.g. after an instance of exercise has been completed.

The second category of data may be processed at least in part by the module processor. The second category of data may be processed by a further processing device arranged to communicate with the module during exercise, e.g. within a local wireless network.

The third set of data may be processed by the processor of the module and/or one or more remote processor, computer or computing/server system. The remote system may comprise a data store comprising historic sensor and/or exercise parameter data.

The electronics module may comprise a data output/communication circuit, such as a transmitter. The electronics module may transmit either, or a combination of, raw sensor data and processed sensor data for processing/analysis remotely of the electronics module.

The optional features defined herein in relation to any one aspect of the invention may be applied individually or in combination to any other aspect of the invention wherever practicable.

### BRIEF DESCRIPTION OF THE DRAWINGS

Practicable embodiments of the invention are described in further details below by way of example only with reference to the accompanying drawings, of which:

FIG. 1 shows a side view of an electronics housing module according to an example of the invention;

FIG. 2 shows a three-dimensional view of the housing of FIG. 1 from above;

FIG. 3 shows a view of the underside of the housing of FIG. 1;

FIG. 4 shows a schematic view of the basic electronic components of a housing module according to an example of the invention;

FIG. 5 shows a plan view of a harness with the electronics module attached thereto prior to positioning on a wearer;

FIG. 6 shows a three-dimensional view of an electronics housing being applied to a harness located on a wearer's body for use according to an example of the invention;

FIG. 7 shows a schematic overview of the processing of sensor data according to an example of the invention;

FIG. 8 shows an example raw sensor signal data plot for an accelerometer;

FIG. 9 shows an example signal plot for fused sensor data showing angular orientation, i.e. roll;

FIG. 10 shows an example of identification of individual cycles for a cyclic motion and determination of an effectiveness indicator for each cycle;

FIG. 11 shows a wider computational system for reporting and/or analysis of data sensed according to an example of the invention; and

FIGS. 12-15 show different examples of sensor network topologies adoptable according to different implementations of the invention.

### DETAILED DESCRIPTION

The invention derives from a need to accurately assess key performance indicators (KPI's) that contribute to technique and performance in competitive sporting activities, particularly activities where a cyclic/periodic motion contributes to performance, such as swimming, running, cycling, rowing and the like. The invention aims to provide accurate, quantitative information to aid training decisions and monitor progress for athletes, such that it can be used to drive behavioural change for individuals or multiple members of a team. The invention may facilitate a data-driven approach to training, e.g. for a broad user base rather than individual elite athletes.

### Module and Housing

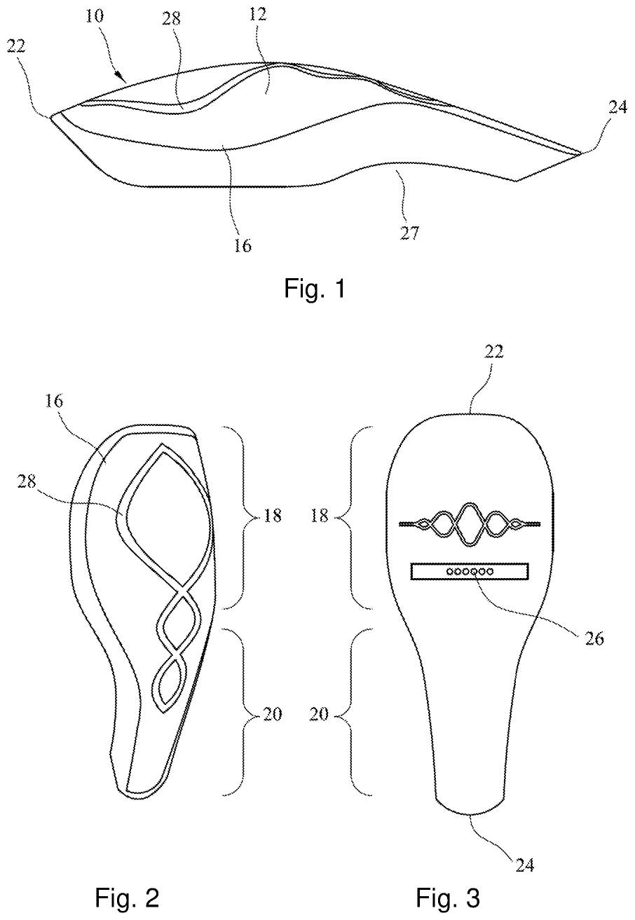

Turning firstly to FIGS. 1-5, there is shown an electronics housing module 10 comprising an outer shell-like casing structure 12 providing a solid outer wall enclosing an internal enclosure/space 14 in which electronic devices are mounted. The casing 12 in this example is waterproof and may be formed from two opposing casing sections that are brought together and sealed along an interface/join 16.

The casing 12 has a more bulbous head portion 18 and a narrower neck/tail portion 20 in plan and may be generally teardrop-shaped. This shape is well suited to use in swimming, amongst other sporting activities, due to its hydrodynamic, aerodynamic and/or unobtrusive form. The profile shown reduces drag and avoids recirculation/stagnation regions of flow over the casing in use. However the tapering tail portion 20 also allows location of the module on the upper back of a wearer, i.e. with the tail between a wearer's shoulder blades, with minimal disruption to the wearer and without limiting the wearer's full range of movement during exercise. The specific location of the module in this region during use will be discussed in more detail below.

The casing 12 has a smooth upper surface which is curved in profile, e.g. in both longitudinal and lateral profile. The upper surface reaches a maximum height part way along the length of the module 10 and curves downward towards its front and rear ends. The upper surface reaches a maximum height towards the centre of the device in a lateral direction and slopes downwards towards its lateral edges.

A front edge of the casing 12 provides a tip or nose, i.e. a leading edge formation 22. The underside of the casing slopes in an opposing sense (i.e. upwards) from the slope of the upper surface towards the leading edge 22, thereby forming a tapering/narrow leading edge. The trailing edge 24 may comprise a similar tapering formation.

The underside of the casing 12 comprises an electrical connector formation 26 part-way along the head portion 18. The connector 26 in this example is recessed into the casing but could otherwise comprise a projecting formation. The connector is typically a multi part/pin connector 26 and may comprise a conventional USB connector/interface. The connector is arranged for releasable connection with a harness connector to be described below and/or may be used for charging the module.

The underside of the head portion 18 is generally flat/planar in form and is arranged to sit on the harness and generally flat on the wearer's body/skin in use.

The underside of the tail portion in this example is arched/raised to form a gap 27 in use beneath the casing and the wearer.

On the upper/outer surface of the casing 12, there is provided a distinct visual pattern 28, thereby providing visual indicia to an observer. The indicia is a contrasting/lighter colour than the remainder of the upper surface. The combination of the pattern 28 and the curvature of the upper surface means that the indicia is identifiable from a wide range of viewing angles. The pattern covers an area of the upper surface, e.g. extending over a portion of the head 18 and tail 20 regions of the casing.

The pattern 28 may comprise one or more curved waveform. In this specific example, the pattern 28 comprises overlaid, opposing and/or mirrored waveforms, e.g. of the same or differing magnitude. This pattern spans a relatively large area of the casing surface and is easily perceived by an onlooker or camera.

The underside of the casing may comprise a textured/friction surface, whereas the upper surface may be smooth. Two different components having said different surface properties may provide the respective upper and underside surfaces and be brought together to define the casing.

Although not shown in the figures, a simple user control, i.e. a button, may be provided on the casing exterior, for example on a sloping region of the casing underside, such that it is not accidentally depressible, is in easy reach and can be blindly activated by the user when mounted. The button may comprise a power on/off button and/or a reset button. Different functions may be attributed to different press durations of the button, e.g. a press-and-hold input being used for power on/off.

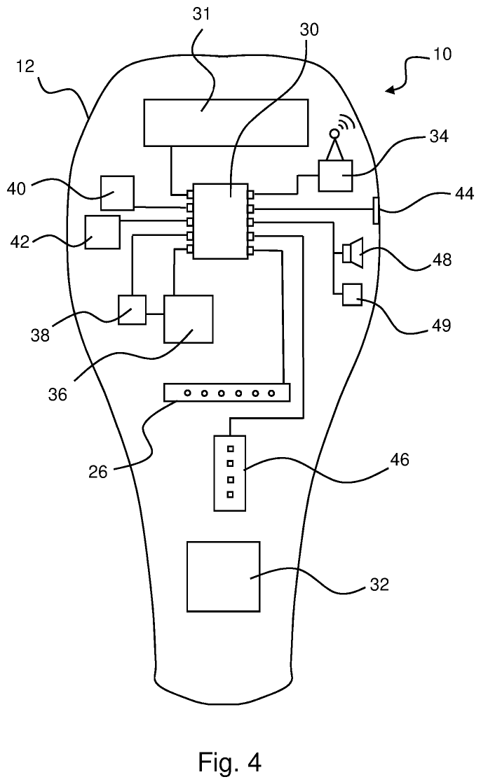

Turning now to FIG. 4, there is shown a schematic of the electronic components of the module 10. The module comprises:
one or more programmable processor 30, e.g. one or more chip;
an on-board non-volatile data store 31
a power source 32, e.g. in the form of a rechargeable battery
a data output device, e.g. wireless signal transmitter 34
a number of sensors 36-44
one or more feedback device 46, 48, 49
electrical connector 26

The processor 30 comprises a central processing unit (CPU) for the module having multiple inputs and outputs and operating as a controller for the module as well as performing sensor data processing operations.

At the core of the module's sensing capability is motion sensor 36. The motion sensor typically comprises one or more multi-axis, e.g. 3-axis, motion sensor such as an accelerometer or gyroscope. In the present example, the motion sensor comprises a multi-axis inertial measurement unit (IMU) having each of an accelerometer, a gyroscope and a compass sensor which are used collectively to provide orientation, position and acceleration data. In other examples, a magnetic field sensor is used to provide orientation data, e.g. a two or three dimensional magnetometer.

Each sensor of the IMU has at least two degrees of freedom (giving a 6-axis IMU) and preferably has three degrees of freedom, thereby providing a 9-axis IMU. However it is noted that in other examples, it may be preferable to use an IMU comprising an accelerometer and gyroscope but not an integral compass/magnetic sensor. A separate compass/magnetic sensor or magnetic field sensor could still be used if required as an optional additional device. Thus the core IMU could comprise a 6-axis IMU having two three axis-sensors, such as accelerometer and gyroscope sensors.

The different motion/orientation sensors may be mounted on a common board and/or may have a controller/microcontroller/microprocessor (i.e. a dedicated controller separate to processor 30) which manages the different sensor inputs to provide a combined, series and/or processed output, e.g. in real time. Whilst the sensors 36-44 are all shown as being separately connected to the processor 30, in various embodiments at least some sensors are provided in combination with a chip, i.e. separate from processor 30, for performing initial collation, time synchronisation and processing of the received sensor signals. The processor 30 may then receive the initially-processed or fused sensor data output signal for processing to determine performance parameters for feedback to a user as will be described below. In other examples, the processor 30 could be programmed to accommodate the raw sensor inputs from the different sensor types making up the IMU.

Suitable algorithms for combining the IMU sensor operation and/or outputs are provided on the relevant processor/controller. Problems associated with IMU sensor data, e.g. high noise levels and additive bias, have been noted in the art and suitable algorithms proposed, such as the filtering solution disclosed by Mahony et al, IEEE Transactions on Automatic Control (Volume: 53, Issue: 5, June 2008). A suitable orientation algorithm for a wearable IMU is disclosed by Madgwick et al, Estimation of IMU and MARG orientation using a gradient descent algorithm, 2011 IEEE International Conference on Rehabilitation Robotics (ICORR). The various techniques available for such purposes will not be described herein in further detail for brevity since general-purpose examples are available to the skilled person.

Additional sensors on board the module 10 comprise any, any combination, or all of:
an internal temperature sensor (IMU temperature sensor 38 and/or CPU temperature sensor 40)
an internal humidity sensor 42
external/ambient pressure sensor (i.e. barometric pressure sensor) 44
a global or local localisation sensor (i.e. GPS or GNSS)

The temperature, humidity and/or pressure sensors may be used to monitor the working condition of the module 10, i.e. as separate from the sensors that contribute to the performance monitoring of the athlete. In various examples of the invention different arrangements of module condition sensors may be used to monitor correct operation of the module 10.

The processor 30 in general terms will receive sensor and/or control inputs in use and process them in order to generate performance data and/or control signal outputs.

The output devices under the control of processor 30 comprise any, any combination or all of:
wireless communication device 34 offering short range wireless communication send/receive capability, e.g. according to Bluetooth®, WiFi® and/or other suitable communication protocol
audio output device/circuit 48 such as a speaker or audio signal generator
haptic feedback device 49, e.g. a conventional vibration device
a visual output device such as a light. In this example, a plurality of individually operable lights, typically LED's, are provided in an array 46 under the control of processor 30.

In the example shown, an additional location sensing system, e.g. comprising a location/satellite signal receiver and associated processing is not provided since it is superfluous to the immediate aims. However such features may be provided as required for monitoring swimming or other sports, particularly outdoors.

### Harness and Module Mounting

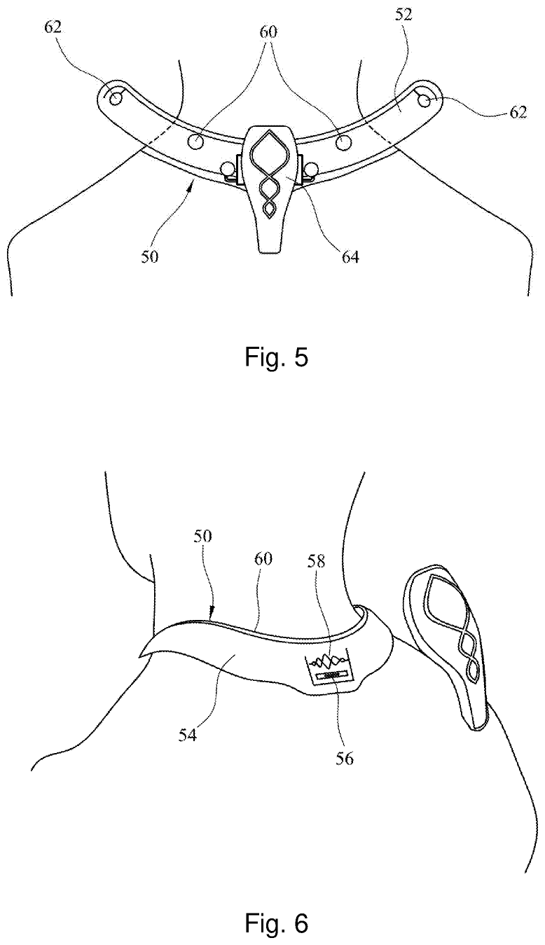

Turning now to FIGS. 5 and 6 there is shown details of a harness component 50 and its connection to the module 10 in use. The harness 50 is provided to facilitate mounting of the module 10 on a wearer's body. The harness can provide a multi-purpose interface between the module 10 and the wearer. The harness may also provide feedback to the wearer and/or an onlooker/coach.

The harness 50 shown in FIG. 5 comprises a flexible substrate 52 arranged to conform to the contour of a portion of a wearer's torso. The substrate comprises a compliant sheet material, e.g. comprising one or more polymer layers. The substrate typically comprises a multi-layer laminate structure such that the interface between adjacent layers may allow sensor and/or feedback devices to be mounted/embedded within the structure of the flexible substrate. Electrical conductors are also located and/or encapsulated between adjacent layers of the substrate 52.

In various embodiments of the harness, different layer arrangements may be used, comprising at least one insulating layer, a conductive layer for electrical connection of components and potentially one or more shielding layer.

The harness 50 as shown in FIG. 6 comprises an additional outer layer 54 over the substrate 52 so as to provide a cover. The outer layer could comprise a polymer/elastomer material or else could comprise a textile material layer.

The material of the substrate 52 may be flexible not only out of the plane of the substrate but also due to elasticity of the substrate material, i.e. within the plane of the substrate. This may improve the manner in which the harness 50 can cling to the wearer in use.

In various examples, the substrate 52 and/or outer layer 54 could comprise an elastomer, such as a fluoro-elastomer material.

The harness 50 comprises an electrical connector 56 for coupling to the connector 26 of the module 10 in use. The region around the connector 56 is arranged to cooperate with the underside of the module 10 and may comprise a cradle-like structure or friction surface portion against which the module 10 can be seated. In this example, the harness comprises a shallow recess 58 which receives the head portion 18 of the module 10.

In various examples of the system, the module 10 and/or harness 50 could comprise a latch, clip or other releasable fastening structure to couple the module 10 onto the harness 50 for use. The electrical connector could comprise a spring-loaded latch or other projection for this purpose. A different type of releasable push-fit connector could be used either integrated with the electrical connector or as a separate mechanical connector. The connector could be provided with a one-way mechanical key structure, shape, or alignment formation i.e. such that the user cannot attach the module to the harness in an incorrect orientation.

In specific working embodiments of the invention, the connector comprises a releasable mechanical connector/cradle having one or more latch members arranged to couple the module 10 with the harness in use. Resiliently biased latch members may be provided to engage with openings in the underside of the module 10 housing. Opposing actuators on either side of the cradle may be used to release the module 10 from the cradle, e.g. in the manner of a ‘pinch-to-release’ or ‘click-to-release’ mechanism. A positive mechanical coupling has been found advantageous to ensure sure and accurate positioning of the module 10 in use.

The harness may comprise an adhesive, e.g. an adhesive region or layer, on its underside for adhering the harness 50 to the wearer. An adhesive film may be used.

In some examples, replaceable silicone-gel adhesive pads are used to secure the harness to the user. A medical-grade, skin safe, hypo-allergenic, high-tack silicone gel may be used. A suitable adhesive layer/pad may be reusable and/or repositionable, i.e. peelable. Suitable adhesives have been found that are compliant with hair and capable of maintaining robust adherence with skin when in contact with water. When the module is mounted on the harness, it can be seen that the casing 12 extends beyond the perimeter of the substrate 52. In this example the harness 50 takes the form of an elongate strip and the module extends substantially perpendicularly to the longitudinal axis of the harness.

The harness 50 extends on either side of the connector 56 to provide opposing limbs depending outwardly from the module 10, when mounted. The harness in this example is arched, which the mount for the module 10 being at the centre of the arc. The centre of the harness may provide a reference feature for alignment with the wearer's spine.

The harness in this example takes the form of a flexible yoke/collar structure arranged to extend over a wearer's shoulders and part-way around a wearer's neck as shown in FIG. 6. This allows the module 10 to be securely positioned on the wearer's upper back region such that the module is aligned with the spine.

This positioning has been found to be particularly beneficial for the module 10 since it can detect movements, e.g. arm movements, for each of the left-hand and right-hand sides of the body independently, as well as pitch, yaw and/or roll for the torso and movements of the head.

In use, the positioning of the module has been found to be highly beneficial for obtaining cleaner/holistic movement data and for more accurate derivation of forward velocity of body (trunk) as will be described below. The upper spine location provides highest quality data for swimming in particular to aid reliable, detailed metric extraction and insight.

The location of the harness/module is unobtrusive to the wearer and allows inspection of the module/harness by an onlooker.

The positioning of the harness such that it extends over a wearer's shoulders has been found beneficial in that it does not interfere with free movement of the wearer in any way and the harness provides a mechanical/weight-bearing property, akin to hanging from the shoulder. When used for swimming, the harness and module have been designed specifically with hydrodynamics in mind such that the profile is contoured to reduce drag and produce down-force by way of the flow over the casing and at its edges. The module therefore presses down slightly against the wearer's skin during forward motion through water and avoids hydrodynamic lift that could compromise adherence of the harness to skin. The frontal facing portion of the harness over the wearer's shoulder avoids edges of the harness facing the flow direction, i.e. reducing the likelihood of flow entry under a leading edge of the harness that could serve to initiate unwanted peeling of the harness.

The specific harness and module arrangement removes the likelihood of chafing, constricting belts, buckles or straps. The harness is contoured also to increase comfort for the wearer.

Other examples of harnesses compatible with this invention include different types of wearable support/mounting structure such as straps, bands or textile-based garments akin to a sports bra, swimsuit, triathlon suit, wetsuit or training top. Whilst the specific harness of FIGS. 5 and 6 is particularly suited to swimming amongst other sorts, close-fitting garments may provide an adequate solution for many activities and performance-monitoring scenarios. The term ‘harness’ as used herein is to be construed accordingly.

Each of these examples of harnesses include various additional sensors and/or feedback devices specific to their application. The close fitment or adherence of the harness to the intended location on the wearer's body/torso is an important consideration as well as the desired location of the sensor(s) or feedback device(s). Garments, textiles or adhesive patches capable of meeting such design considerations may all be considered in further embodiments of the invention. Various techniques to sew/stitch or otherwise embed the electrical connector 56 and/or a mechanical cradle structure, either with or without sensors or feedback devices and associated electrical conductors/wires, into a garment may be considered.

Electrical connection with the module 10 allows the components of the harness to be powered by the module power source and also allows signal communication between the module 10 and harness 50, i.e. for sensor signals to the module and/or feedback/output signals from the module. The connection allows real-time feedback functionality using the embedded electronics of the harness.

In the manner described herein, the module 10 is a self-sufficient module, having a core sensor capability, that can be used either with or without a harness connected thereto. The harness 50 in contrast is dependent on connection with the module 10 for operation and is used to provide additional sensing and/or feedback capabilities to those of the module. The electrical connection may thus provide a power and/or data communication interface. The harness may accordingly comprise a plurality of electronic components which may be generic or else adapted to the exercise/sport being monitored. In particular, the substrate of the harness may comprise a plurality of sensors, e.g. of the same or different types, and/or a plurality of feedback devices of the same or different types.

When the module 10 and harness 50 are connected, the module performs a handshake process to verify the identity and/or makeup of the harness. The processor 30 may thus determine the compatibility of the harness with the module 10 and the additional sensing/feedback functionality of the harness. This may determine the anticipated input/outputs for the processor 30 in use and which data processing algorithms/modules will be required to accommodate the connected harness.

Technical features of the harness comprise any, any combination, or all of:
One or more visual LED array 60, e.g. on the left and/or right side of the harness. The array 60 may comprise a strip and/or may offer varying colour/RGB output and/or lighting patterns. The processor 30 may control the number of lights lit and/or colour of illumination as an indicator of a variable parameter value being monitored.
One or more haptic (vibration) motor 62 on left and right sides
Auditory feedback/communication device 64. The device 64 comprises an audio output device/circuit such as a speaker or audio signal generator. In some examples, the device 64 could use bone conduction, e.g. comprising an audio vibration generator for communication with the spine/skull.
Optional additional motion sensors (e.g. IMUs) for functional extension of the module sensors
Optional additional location-based sensors (e.g. GPS, GNSS) for additional data input to be combined with the module sensors
One or more physiological sensor (e.g. such as heart rate, blood oxygenation, or other). In this example an optical heart rate sensor is provided.

Additional or alternative features of the harness may comprise: one or more sensor for monitoring an external/ambient environmental condition; an additional wireless communication device/antenna, e.g. for Bluetooth (including Bluetooth Low Energy or Bluetooth Smart), WiFi and/or near field communication; and/or a GPS receiver or other tracking device.

Using the above features, the harness acts as a modular extension of the module's functionality by providing additional measurement, communications and/or feedback options for users.

Where additional sensors are provided on the harness, the system may establish a local Body Area Network (BAN) for communication between the relevant sensor and processing components.

### Motion Sensor Data Processing

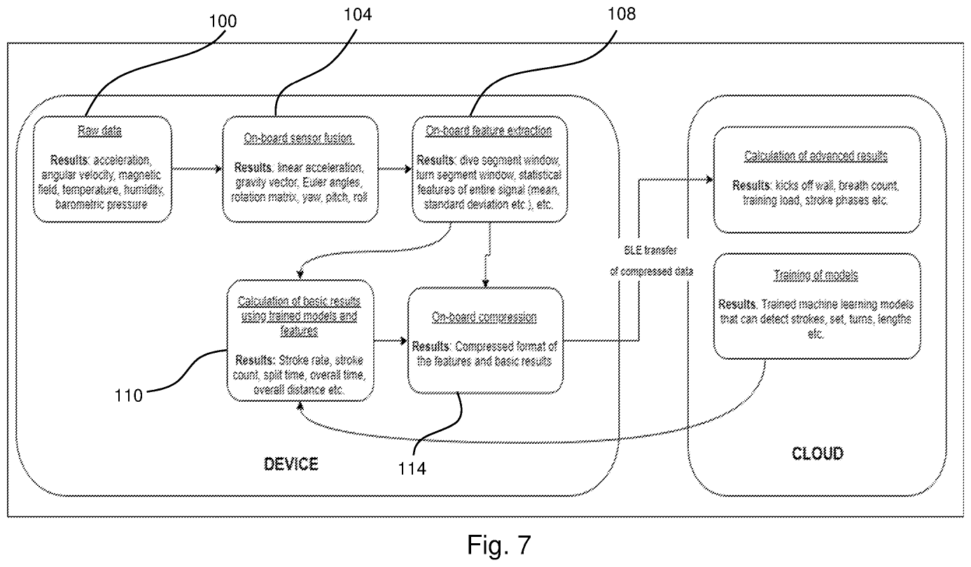

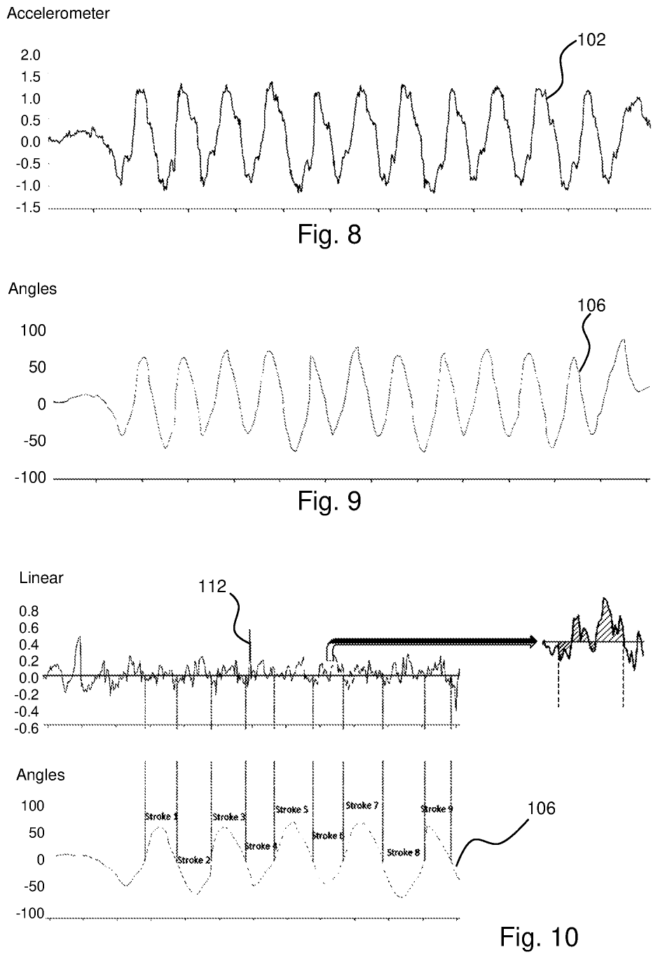

Turning to FIG. 7 there is shown an overview of the data processing stages during operation of the various examples of the module 10 described herein. The raw sensor data from the sensors used for performance monitoring is received at stage 100. In this example the results comprise readings of multi-axis acceleration, angular velocity and orientation data (e.g. in the form of magnetic field readings). The readings are captured over time and recorded using a common clock such that the recorded signals are all captured in a time synchronised manner. An example of the raw signal 102 from an accelerometer over time is provided in FIG. 8.

Sensor data/signal fusion is performed on the received/raw sensor signals at stage 104, which may be described as a data fusion module. The fusion process involves processing combinations of two or more raw sensor signals to generate resulting signals to be used for feature extraction and performance monitoring. In general terms, the data fusion process involves processing two or more sensor signals together to improve the accuracy or usefulness of the output.

The sensor data capture and/or fusion process may be synchronised at a predetermined frequency, such as at 50 Hz in this example. Conventional static and/or dynamic calibration techniques can be used as required.

FIG. 9 shows an example of one output signal of the data fusion process 104 in which a signal/plot 106 of the roll angle over time is generated. This may be produced by vectorising (e.g. using a vector component of) acceleration and angular velocity sensor signal inputs.

Additional or alternative output signals that may result from the fusion process comprise: a linear acceleration signal (i.e. derived from the received total acceleration signal); a gravity vector; and/or angular orientation signals about individual axes, i.e. for pitch and/or roll. Euler angles and/or a rotation matrix may be generated using the orientation data relative to the different axes. Data fusion may be used for a plurality of reasons. For example, where sensors, e.g. such as a gyroscope, determine relative values that can be subject to potential drift errors over time, the fusion process can be used to mitigate such potential errors. In other examples, it may be desirous to determine not only a linear acceleration relative to the orientation of the device in use, but also an earth acceleration signal (i.e. relative to a global reference plane, such as horizontal/vertical). In such examples where one or more component of a signal in a different reference plane is required, a gravity vector of the accelerometer, or other orientation signal component, can be used to process the signal.

In the example shown, the stages 100 and 104, and associated modules for said stages, may or may not be comprised in an initial processor/chip, the output of which is communicated to the processor 30.

The on-board processor 30 performs data feature extraction at stage 108, whereby the specific features and parameter values to be used by the performance monitoring function are determined. Extracted features may for example may comprise zero crossings, max/min points, rapid changes in gradient, points or zones for which one or more threshold is exceeded, and the like. Any or any combination of such features may be used to identify cycles or a cyclic motion performed by the wearer, and thus the amplitude and/or frequency of the cyclic motion, amongst other data attributed to individual or collective cycles.

For example, once individual cycles have been identified, statistical analysis of the data for each cycle and/or a signal as a whole can be performed. Average values of cycle frequency, magnitude and the like can be determined, as well as variation therefrom for each cycle.

Additionally or alternatively, segments of signals can be identified as corresponding to certain phases of an exercise or activity. For example in swimming, a dive, a turn and/or a push-off can be characterised as separate segments/events which should not contribute to the analysis of metrics for normal swimming strokes. Events such as these can be identified and isolated for separate analysis by way of identification of the corresponding signal features, during feature extraction. Isolation of those events/segments also allows analysis and performance monitoring of those events separately from the normal cyclic motion analysis. Repeated segments can be compared using statistical analysis or using any other performance monitoring techniques described herein.

Using the extracted feature values, the processor 30 can generate at stage 110 immediate results on-board the device that can be output using any of the means disclosed herein as feedback signals. These may correspond to any or any combination of cycle rate, cycle count, segment/split times corresponding to distance travelled or cycle count, total duration, total distance travelled and the like.

One benefit of the embodiments described herein is the ability to analyse individual components of motion per cycle and to correlate them, individually or in combination, to a performance metric for the activity in question. For example, individual components of body movement such as rotation (pitch, roll, and yaw) can be assessed in terms of the relative contributions to forward speed or acceleration. This ability to identify individual components of motion and track them relative to each cycle and/or the effectiveness of each cycle is particularly important to the deeper understanding of technique offered by the present disclosure.

One example of a specific metric that can be used for assessment of technique is described in relation to FIG. 10. In the lower portion of FIG. 10 it can be seen how the zero crossing points of the roll angle signal 106 can be used to identify individual cycles, i.e. the start and end of individual swimming strokes in this example. The synchronisation of the relevant sensor signals means that those start and end points (i.e. the delineation points between cycles) can be transposed onto other recorded signals.

In the upper portion of FIG. 10, the linear acceleration signal 112 can be discretised into the individual cycles/strokes using those start and end points. The area under that signal 112 plot, i.e. the area bounded by the signal and the X axis, can be determined per cycle. This provides a very useful tradeable/comparable performance measure relating to one-dimensional motion (e.g. forward motion in this example). This measure is referred to as ‘impulse’ by the applicant and provides a relative net change in speed, i.e. a forward speed gain or loss, for the individual cycle.

It is noted that the impulse value described herein need not be an absolute value and that a relative change in this value can be used to compare different cycles, or contributions to those cycles. However a datum point can be used if it is desired to convert this to an absolute value, i.e. by using a known fixed distance of travel. A suitable datum point can be obtained via various means, including a GPS signal or a known distance of travel, e.g. according to a length of a swimming pool or similar. A turn, or other event at a known distance may be used to determine the datum point.

Whilst the above description refers to determination of impulse values for individual cycles, a similar approach could be used for a plurality, or set, of cycles and/or identified segments. Thus components of the exercise in question can be separately compared to other corresponding components as required e.g. for one athlete or between different athletes.

This process of breaking down the recorded motion to individual cycles and/or segments and determining a relative change in speed, acceleration or other performance metric for each of those cycles/segments by reference to a corresponding time period of another sensor/fused signal output has been found to be beneficial in assessing individual components of technique. Indicators of such assessments can be fed back to users or coaches, e.g. in real time, using any of the feedback means described herein and/or communicated to a remote processing facility for further analysis. Feedback on particular components of technique during training is particularly beneficial for athletes, rather than simpler measures of timing, stroke/cycle count or the like. For example, a dedicated feedback for rotation during swimming can indicate when the degree of roll, pitch or yaw during a stroke is beneficial or detrimental to forward speed.

Dedicated feedback for all kinds of aspects of technique, i.e. the contributions of individual components of motion to a suitable performance parameter, can be provided to an athlete or coach by way of audible, haptic and/or visual feedback in real time during training. Thus an athlete can experiment with individual aspects of technique and get feedback thereon in a way that has not been hitherto possible. The timing of such feedback to an athlete can be crucial in determining whether changes in technique are helping or hindering performance. For example, audible beeps, haptic buzzing or a flashing light can indicate acceptance thresholds for the individual component being monitored or an improvement/reduction in the performance parameter associated therewith.

Whilst the processing steps described above use roll angle as an indicator of the periodicity of cyclic motion, it will be appreciated that for other sports, a different sensor signal or fused signal output may provide a better indicator. For example a component of acceleration or motion in a vertical or horizontal plane may be used or other signal in which the cyclic nature of the motion is most apparent.

Turning back to FIG. 7, at stage 114 the processor 30 collates data/signals for communication off the module for further processing and analysis. The processor could communicate raw data but it has been found beneficial instead to communicate the features extracted at stage 108 and/or results from stage 110 for subsequent processing remotely of the device 10. Compression of the data for transmission is performed as part of stage 114 in this example.

### Wider System

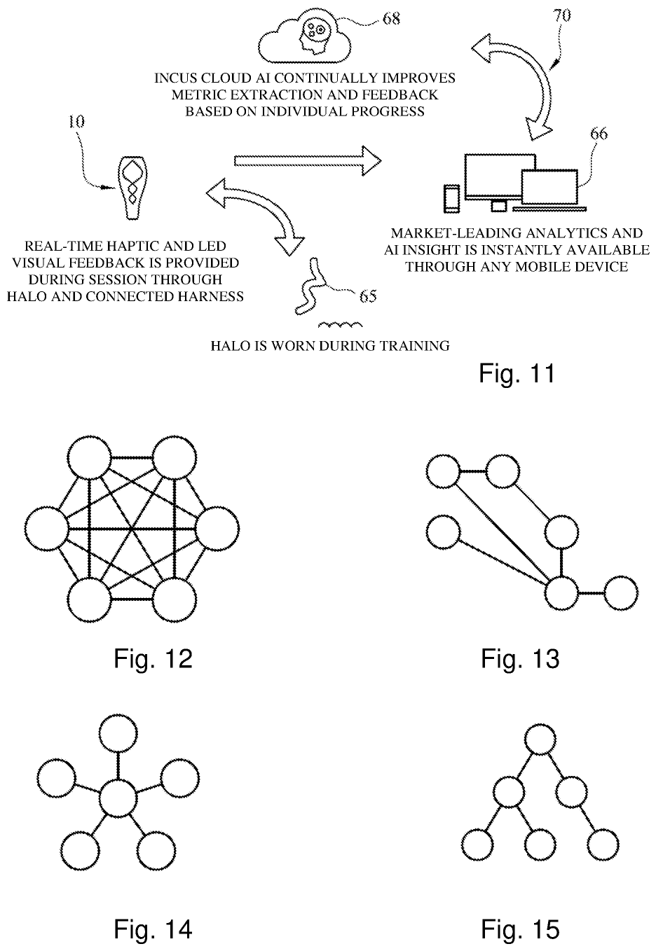

With reference to FIG. 11, a wider monitoring and feedback system 70 comprises one or more module 10 and associated harness 50 arranged to be worn by a user 65, a separate local computational device 66 (such as a mobile telephone, smartphone, tablet computer, laptop, PDA or local PC), and a remote server system 68. The three different devices 10, 66 and 68 making up the wider system allow for three levels of reporting/feedback and/or three associated reporting/feedback timeframes. In different examples, either of the local computational device 66 or remote server system 68 could be avoided and all subsequent data processing could be performed on one of those two data processing devices.

The device 66 is typically a portable computing device. The device 66 has a receiver, data processor and a visual display/screen. The device 66 is running a software application dedicated to the handling and display of information pertaining to the exercise being monitored by the module(s) 10. The device 66 thus offers additional processing and information output over and above the information derived by the module processor 30.

Where the timeframe of data/signal output by the processor 30 of module 10 is real time, the timeframe for the device 66 is referred to herein as ‘immediate’. The device 66 may output summary totals for sets of exercises or the like and associated derived information to be reported during or else at the end of an instance or interim period of exercise. The device 66 may be used by a coach to analyse current performance of one or a number of athletes in a training session. Whether or not a coach is present, an individual user may check performance at breaks between sets of a current instance of training or immediately thereafter using his/her own device 66.

The server system 68 provides deeper processing and analysis for post-session reporting, e.g. operating under a third level of information or timeframe of output. The wider monitoring system is tailored to provide relevant feedback to the athlete(s) and/or coach(es) at appropriate timeframes within the usage cycle of the system. This ensures that insight is most effective to each user, be it an athlete, coach, manager, assessor or other party, e.g. without an overload of extraneous information. Examples of the later insight that can be generated for swimming are given in the right hand side of FIG. 7.

The distinct time-frames of reporting are also used to optimise data transmission and processing efficiency based on feedback requirements for each timeframe. This can help to reduce latency, power consumption, and to overcome complexities of data transfer within certain exercise scenarios, e.g. aquatic environments.

Data is transferred from the local device 66 to the server system 68 using conventional data communication channels, e.g. typically including cellular networks and the worldwide web. The data, or a subset thereof, may also be stored using the device 66 software application, e.g. in the local device data store.

The server system hosts a cloud-based analytics engine that is accessible to registered users, e.g. by remote login, to access current and historic exercise information. This provides a richer data set and analysis tools to the user, e.g. offering a wider variety of user tools for data mining and reporting within the user interface. The server system hosts an online analytics engine.

The data processing by the server system is differentiated from the module 10 and device 66 processing in that it is more computationally complex/expensive and uses proprietary AI algorithms to provide highly-detailed and actionable training feedback. In the present example, the server system data processing code/algorithms allow adaptive learning from data sets to improve performance metric extraction accuracy/robustness for each user, and/or to identify complex performance trends, e.g. including long-term metric or data feature interdependencies, that relate to each athlete or groups of athletes. It is using these tools that improved, actionable feedback is produced, through analysing broad data sets to understand which KPI metrics are most significant to each user's performance.

In the case of swimming, the server system can potentially analyse more than twenty-two unique performance metrics from a swimmer's stroke and relate them to forward speed over time. From this, it can suggest which technical aspects are most crucial to work on for overall performance benefit. The server system may output a ranking for each unique performance metric based on analysis of the impact of each metric on one or more overall assessment criterion, such as forward velocity, turn duration, or the like.

For each analysis/reporting device 10, 66, 68 of the system 70, an example breakdown of the functionality is provided below:

### Real-Time

Definition: Processing and/or feedback to the athlete whilst expending effort during the activity/training set

Method: Haptic (vibration) and/or visual LED and/or auditory feedback to the user by module 10 or harness 50 based upon on-board data fusion and processing

Example swimming/activity metrics comprise: Speed zones, stroke pacing, stroke type, heart rate zones, left/right side power symmetry, push off velocity and/or start sequences.

### Immediate

Definition: Local reporting between sets of a common instance of activity, e.g. when the athlete is recovering and/or preparing for the next set

Method: Raw and/or semi-processed data wirelessly uploaded from module 10 to a mobile device (when available) for further processing and viewing via mobile app

Example swimming/activity metrics comprise: set durations, split times, forward velocity over set, power symmetry over set, stroke phases, turn efficiency, breathing statistics, vital sign (HRM) traces over set and/or start reaction times

### Post-Session Processing

Definition: After a training instance has finished and/or away from the training environment, typically in a remote computational environment

Method: Full data uploaded to cloud server system and processed using AI algorithms. Extended insight made available for display on mobile device via software application or online portal/web pages accessible to users

Example metrics comprise: training load, actionable AI insight, athlete comparisons, historic performance/progress charts, season/strategic planning, session planning and/or athlete selection.

Real-time processing of the received sensor signals on-board module 10 by processor 30 allows determination and output of feedback by output devices of the module and/or harness. The colour and sequence of illumination of LEDs in the lighting array 46 or 60 correspond to signal processing output, e.g. derived exercise parameters, on-board the module. This allows current assessment by an onlooker/coach and/or accurate synchronisation with vision systems, such as high speed vision cameras and associated processing means.

Illumination colour and/or sequence (motion) of LEDs can be used to indicate various exercise indicators/parameters or other information. The parameters used for feedback can either be specified by the user, e.g. via the software application on local device 66 or can be one or more default setting of the system. The parameter used for feedback could comprise any or any combination of forward speed, stroke power, stroke rate, heart rate, blood oxygenation, turn time, breath duration, etc. The feedback could additionally or alternatively be used to indicate an operating state of the system (e.g. on/off, collecting, idle, transmitting data, low power).

Turning now to FIGS. 12-15, the system may adopt an adaptive network topology for communication amongst components of a body area network or multiple local modules 10, e.g. being used as part of group training/competition. Local device(s) 66 can typically be included in the established local network. Adaptive networking procedures may implement a topology that optimises communications based on: environment measurement; communication bandwidth availability and combination/number/availability of units and harnesses deployed in a training session

This adaptive networking is used to overcome complexities of wireless data transmission where obstructions to data communication may occur, e.g. transiently. Such considerations can occur due to submersion in water, signal interference in groups of users, obstructions due to scenery, equipment, group users or other people, and/or for synchronised multi-segment measurement.

The system will use and/or combine network topologies such as:
Body area Network (BAN)/Wireless Body Area Network (WBAN), e.g. between harness and module or components thereof, such as embedded physiological/motion sensors.
Full/partial mesh networking between devices
Star/Tree networking between devices
   to economise data transfer, synchronise multiple units and manage data communications. The system will automatically select the correct/best network topology for the current instance of use.

In some examples, network topology and communications can be used to reduce drift errors typically associated with IMU sensors. That is to say by monitoring of communications with other devices in the network, e.g. in a mesh network, processors 30 can determine whether and to what extent an individual IMU is encountering drift error. The readings from other devices can be used to establish datum points or thresholds for identifying and/or correcting IMU drift.

The sensors of the module and further sensor(s) of the harness may collectively form a mesh network.

Full and partial mesh network topologies are shown in FIGS. 8 and 9. Each node/device relays data for the network, i.e. to one, some or all other nodes, and so mesh nodes cooperate in the distribution of data within the network. Such networking is highly scalable and able to adapt to temporary unavailability of nodes. Example scenarios/functionality for such networking include:
Communication between multiple modules 10 to extend effective data transfer range to a coach mobile device/tablet if conventionally out of range
Quick and efficient data transfer to all modules 10 in a group from a single device, e.g. the coach's device 66. This could be used to send instructions or settings changes, such as a start data capture command for all modules, or a change feedback setting change for all modules.
Synchronous, multi-segment monitoring of an athlete wearing multiple measurement devices or multiple athletes
Improvement of the system's spatial understanding by relaying time signatures to other athletes/modules (e.g. triangulation)

For the star and tree network topologies of FIGS. 10 and 11, every node is connected to a central/primary node, which acts as a conduit for data transmission

Example scenarios/functionality for such networking include:
Submerged or obscured measurement devices relay data to a more communicable central node, which may be automatically selected through signal strength/connectivity hierarchy
Connection of body area network devices to connect to the local monitoring device 66 and/or rest of group network from a single ‘lead’ node
In a large, close group (e.g. peloton cycling or triathlon swimming) where interference is a key transmission issue, the system automatically groups and selects a ‘lead’ module (based on signal strength/position), through which other locally networked devices route signal transmissions. This can act to reduce external interferences
For spread out groups, where not all modules are within communication range of each other or the local monitoring device 66, communications may be routed to the lead node to extend communication range
If the local monitoring device 66 has an upper limit on the number of connections (e.g. via Bluetooth), the routing of signals through other nodes allows a larger group of modules to communicate with the device 66, e.g. above its theoretical connectivity limit, without need for a separate base-station to aggregate signals

In view of the above disclosure, it can be appreciated that accurate, quantitative information to aid training decisions and monitor progress is made available to athletes in a way that can improve efficiency/effectiveness at driving behavioural change and also in a way that is practical for multiple members of a team. This addresses the increasing need for accessible, detailed and understandable performance monitoring and analytics in sports such as swimming, where a truly data-driven approach to training is still currently unfamiliar and increasingly important for success.

1. An exercise and performance assessment system comprising:
a harness adapted to be attached to the torso of a wearer;
a performance feedback device;
an electronics module comprising a housing having a plurality of motion sensors; and
a processor for processing received sensor data signals and outputting a resulting feedback signal to the feedback device,
wherein the harness is arranged to hold the housing at a predetermined location on the torso of the wearer and comprises a mount for releasably mounting the module housing to the harness, and
the processor comprises a sensor signal receiver arranged to receive motion sensor data signals over time from the plurality of motion sensors and to log the received signals with a synchronized time signal,
the processor processing one or more of the received sensor data signals so as to identify data features therein indicative of repeated movements by the wearer and to delineate each repetition thereof,
wherein the processor applies the repetition delineation to one or more further received motion sensor data signal in order determine a performance parameter for each individual repetition thereof.

2. The system of claim 1, wherein the motion sensors comprise one or both of an angular orientation sensor and an angular rate sensor.

3. The system of claim 2, wherein the processor identifies the data features and/or delineates between successive repetitions according to rotation of the wearer about one or more axis based upon one or both of angular orientation values from the angular orientation sensor and angular rate values from the angular rate sensor.

4. The system of claim 1, wherein the processor determines a relative linear acceleration or velocity signal from the received motion sensor signals and determines the performance parameter based upon the relative linear acceleration or velocity signal.

5. The system of claim 4, wherein the processor determines an area beneath the relative linear acceleration or velocity signal.

6. The system of claim 1, wherein the processor identifies statistical data features for each successive repetition.

7. The system of claim 1, wherein the processor generates an output signal based upon the performance parameter for transmission to the performance feedback device during exercise by the wearer in real time relative to each repetition of the repeated movements.

8. The system of claim 1, wherein the harness comprises an electrical connector for establishing an electrical connection with the electronics module when mounted thereon,
the harness extending over a portion of the torso in use and comprising one or both of the performance feedback device and a further sensor for communication with the electronics module when connected thereto.

9. (canceled)

10. (canceled)

11. The system according to claim 8, wherein the electronics module accesses a harness identifier upon electrical connection with the harness in order to identify one or both of a sensor and feedback device configuration of the harness.

12. (canceled)

13. (canceled)

14. (canceled)

15. (canceled)

16. The system according to claim 1, wherein the performance feedback device comprises an array of individually controllable lights on the electronics module or harness.

17. The system according to claim 1, wherein the harness is shaped so as to provide a collar or yoke arranged to extend at least part way around the wearer's neck in use.

18. (canceled)

19. (canceled)

20. The system according to claim 1, wherein the harness mount is arranged to support the electronics module on an upper torso region of a wearer, for example between a wearer's shoulder blades and/or in the region of the interface between the upper thoracic spine and lower cervical spine of a wearer.

21. The system according to claim 1, wherein the harness mount is arranged to support the electronics module on the sagittal plane of a wearer's body and/or aligned with the wearer's spine.

22. The system according to claim 1, wherein the housing comprises a wider body portion and a tapering tail region.

23. The system according to claim 1, wherein the housing is convex in form on its upper surface and comprises a concave portion on an underside of the housing.

24. The system according to claim 1, wherein an outer surface of the housing comprises one or more light, the one or more light being controlled by the processor to provide one or more of varying colour, brightness and lighting pattern output according to one or more output of the processing of the received sensor data.

25. (canceled)

26. The system according to claim 1, wherein the processor processes the received sensor data according to a plurality of routines or modules, a first routine processing the received sensor data and outputting a resulting feedback signal to the performance feedback device in real time and a second routine processing the received sensor data to provide analysis with a time delay during or after an instance of exercise by the wearer.

27. (canceled)

28. (canceled)

29. (canceled)

30. The system according to claim 1, wherein the motion sensors comprise a plurality of multi-axis movement sensors of different types, including at least two of: and accelerometer; an angular rate sensor; and a direction/orientation sensor.

31. The system according to claim 1, wherein one or both of the electronics module and the harness comprises one or more location sensor.

32. A method of exercise and performance assessment comprising:
attaching a harness to the torso of a wearer;
releasably attaching to the harness an electronics module comprising a housing having a plurality of motion sensors such that the harness holds the housing at a predetermined location on the torso of the wearer;
receiving at a computer processor motion sensor data signals over time from the plurality of motion sensors and logging the received signals with a synchronised time signal;
processing one or more of the received sensor data signals so as to identify data features therein indicative of repeated movements by the wearer and to delineate each repetition thereof;
applying the repetition delineation to one or more further received motion sensor data signal in order determine a performance parameter for each individual repetition thereof; and
outputting a resulting feedback signal based upon the performance parameter to a feedback device.
